(19) United States
(12) Patent Application Publication
(10) Pub. No.: US 2017/0224809 A1
(43) Pub. Date: Aug. 10, 2017

(54) USE OF SUPERANTIGENS FOR IMPROVING MUCOSAL ALLERGEN SPECIFIC IMMUNOTHERAPY IN NON-HUMAN MAMMALS

(71) Applicant: Premune AB, Stockholm (SE)

(72) Inventors: Sofia Östman, Sävedalen (SE); Agnes Wold, Göteborg (SE)

(21) Appl. No.: 15/519,493

(22) PCT Filed: Oct. 9, 2015
(86) PCT No.: PCT/EP2015/073403

§ 371 Date: Apr. 14, 2017

(30) Foreign Application Priority Data
Oct. 14, 2014 (SE) 1451226-3

(51) Int. Cl. A61K 39/35

(52) U.S. Cl. CPC A61K 39/35; A61K 2039/552

(57) ABSTRACT

Use of a superantigen in mucosal allergen specific immune therapy (ASIT) in a non-human mammal to enhance the effect thereof. In order to enhance the effect of the mucosal ASIT, the superantigen is mucosally administered before, or with, the allergen to the non-human mammal.

### FIELD OF THE INVENTION

The present invention relates to mucosal allergen specific immunotherapy aiming at desensitizing allergic non-human mammals toward allergens causing adverse immune mediated reactions.

### BACKGROUND

Allergy denotes adverse immune mediated hypersensitivity to harmless substances present in foods stuffs or in the air, so called allergens. Allergy may involve IgE-mediated, as well as non-IgE-mediated reactions. IgE-mediated reactions to food early in life are often later followed by IgE-mediated allergy to airborne allergens and symptoms such as asthma and hay fever (1).

The prevalence of allergy among cats and dogs are not known, due to the lack of epidemiological studies. Some insight can be gained through studies comparing allergy diagnosis to all other type of diagnoses. In a study of 31,484 dogs in the US, examined by veterinarians at 52 different sites, 8.7% of the dogs were diagnosed with atopic/allergic dermatitis, allergy or atopy (2).

In Sweden, about 80% of all dogs are insured which creates a strong foundation for breed specific statistics on different diagnoses, as all veterinary care events claimed to the insurance company are registered and classified accordingly. Agria Pet Insurance is one of the world's leading pet insurers, currently holding 60% of the insurance premiums in Sweden.

Since 2005, diagnoses relating to allergies and skin problems in dogs have increased by 100% according to statistics from Agria. Recent insurance statistics from the Swedish company, including between 200,000 to 300,000 dogs, demonstrate that today, on average, one of ten dogs, independent of breed, will develop allergic disease. A number of breeds are considered to be high-risk for allergy, including Staffordshire Bullterrier, French Bulldog and West Highland White Terrier, among which up to 45% of dogs will develop allergies (3, 4).

Allergy in dogs is a lifelong disorder, with typical onset reported to be between six months and three years of age. Clinical signs are reported to be uncommon in dogs less than 6 months or greater than 7 years of age (5-7).

Allergy represents a failure of an immunological mechanism called oral tolerance. Normally harmless antigen to which an animal or human is exposed to by the oral or nasal route, will elicit oral tolerance (also termed “mucosal” tolerance) i.e. active suppression of subsequent immune responses to the antigen in question. Oral tolerance has been demonstrated both in humans (8) and experimental animals (9, 10). The mechanism by which oral tolerance is induced remains obscure.

However, a prerequisite for development of oral tolerance is the passage of the dietary antigen across the epithelial layer, a process which renders it tolerogenic. Such “biological filtering” or “tolerogenic processing” was demonstrated already in the 1980s when it was shown that if serum of a fed animal was collected shortly after feeding and transferred to a naïve recipient, the latter became actively tolerant to the antigen fed to the donor (11, 12). Later it was shown by the present inventors that tolerogenic processing involves loading of peptides onto MHC class II within the epithelial cells and export of MHCII-peptide-carrying exosomes at the basolateral side. Hence, such tolerosomes are present in the tolerogenic serum of fed animals (13).

Allergen Specific Immune Therapy (ASIT) is used in the treatment of mammal subjects post the neonatal stage, most typically also post the infant stage, with a mature immune system, including human beings as well as pets, e.g. dogs and cats, suffering from allergic diseases. The aim of the therapy is to reduce the subject's clinical reaction to the specific allergen to which the allergic individual is hypersensitive. This type of therapy has also be denoted hyposensibilization in the art. In ASIT, the subject has previously been exposed to the specific allergen and developed hypersensitive towards it.

ASIT is an effective treatment for allergic asthma and rhinitis, as well as venom-induced anaphylaxis. More recently, oral hyposensitization against, e.g. peanuts, have been tried with promising results (14-25).

In dogs, ASIT has been shown to be promising in alleviating atopic dermatitis. Researchers at University of Wisconsin and University of Florida, have shown that mite-sensitive dogs (26), as wells as dogs (27) experimentally sensitized to dust mites (DM, Dermatophagoides farinae), timothy grass (TG) and ragweed (RW), benefit from sublingual immunotherapy.

ASIT is based upon administration of a specific allergen or a mixture of allergen(s), i.e. the allergen(s) causing symptoms, to the subject first in small amounts and thereafter in increasing doses. The subject will thereby experience an altered immune response. The altered state of immunity in the hyposensitized individual is associated with weaker or absent symptoms after natural exposure to the allergen. Studies have also shown that ASIT may decrease the risk of asthma development and allergy towards additional allergens (28). While ASIT has been shown to reduce the hyposensitivity to allergens such as peanuts, milk proteins and egg, the effect is at the best partial, i.e. low doses of allergen may be tolerated as long as the ASIT is maintained. However, as only low doses of the allergen are tolerated it would be of great interest to improve present ASIT.

Different routes may be applied for the administration of the allergen. Subcutaneous immunotherapy (SCIT) and oral immunotherapy, e.g. sublingual immunotherapy (SLIT), are the most commonly prescribed routes for ASIT and regarding allergic asthma and rhinitis for humans, reduced symptoms and usage of medication and improved quality of life have been confirmed in several double-blind placebo-controlled trials (29). For pets, mainly SCIT has been used.

Both SCIT and SLIT has shown some promise in improving clinical SCORAD scores and lowered the usage of corticosteroids in atopic dermatitis (30). Further, both routes have been proven similar efficacy, but adverse reactions may occur during ASIT.

SCIT is considered sufficiently safe, but the potential for an adverse reaction is always present. In SCIT, reactions may vary from mild to life-threatening anaphylaxis or even death. Because of those safety issues with subcutaneous administration of allergen, very low doses are given initially and the doses are gradually increased with typical intervals of 3 to 7 days between administrations until the treatment dose (maintenance dose) is reached. The treatment dose is the dose regarded as the effective and tolerable. At each subcutaneous administration, the patient has to be under medical observation due to risk of adverse reactions that may require medical emergency treatment. Thus, SCIT demands numerous visits to the clinic for undertaking treatment. Typically, the up-dosing phase last for a period of 3 to 6 months. Once the treatment dose has been reached, the allergen can be administered less frequently, such as monthly to bi-monthly basis. The monthly to bi-monthly vaccination is usually continued for 3 to 5 years. This process reduces the allergic response to allergen exposure—the subject is desensitized to the allergen.

Oral immunotherapy, e.g. SLIT, has been shown to be a safer administration route of ASIT compared to SCIT. The most common adverse events in SLIT are local reactions (oromucosal pruritus or mild local edema) that occur within a few days after administration and that often resolve quite rapidly. Systemic reactions are uncommon after SLIT and fatalities have never been reported (31, 32). As SLIT is considered as safe, the treatment can be executed at home by the patient him/herself without the need of medical observation. However, some allergens are considered to be too potent to safely be used in SLIT as they are associated with severe side effects. Further, current sublingual therapies require frequent, repeated exposures during a long time period, over a year, until any effect can be observed. Measures to improve the efficacy of sublingual immunotherapy, by means of reducing treatment recurrence and timeframe to observed effect is warranted, as the sublingual immunotherapy is safe; anaphylactic reactions has never been reported and it does not require time demanding visits to the clinic.

Most allergens, such as grass pollen, may induce tolerance when administered by either route whereas some, such as venom, need to be given subcutaneously in order for ASIT to be effective. The mechanism of SCIT and SLIT are not fully elucidated, but SLIT is commonly considered as a means to improve oral tolerance. By introducing the allergen by the oral route in a controlled fashion oral tolerance will successively develop and counteract the allergic immune response.

Various examples of oral ASIT include sublingual ASIT, sublingual spit ASIT, and sublingual swallow ASIT. However, all these administration routes represent a controlled induction of oral tolerance towards the selected allergen. Oral tolerance denotes the normal, physiological tolerance induced by proteins that pass across the gut mucosa, including both fed and inhaled antigens (as all inhaled antigens are transported via the muco-ciliary escalator to the pharynx, where they are swallowed). Although the details regarding how oral tolerance is induced are not fully elucidated, it involves processing of the dietary antigen by the intestinal and presumably also the oral epithelium. The result of this processing is the appearance of a tolerogenic form of the antigen, often denoted tolerogen, in serum shortly after the feeding of the antigen. If serum from an animal fed a dietary protein is transferred to a naïve recipient, antigen-specific tolerance to the dietary antigen develops in the recipient.

In successful oral immunotherapy the induction of oral tolerance to the allergen leads to a suppression of the allergic response and hence, a reduction of allergic symptoms. In accordance, an improvement in oral tolerance, by means of improved tolerogenic processing, will enhance the efficacy or oral immunotherapy.

In general, a higher dose of allergen is needed for effective SLIT than SCIT. For example, the dose of grass pollen is 20-30 times higher in each SLIT dose as compared to each SCIT dose i.e. a daily SLIT dose is about equivalent to a monthly dose of SCIT. The efficacy of SLIT and SCIT seem to be similar. As SLIT is regarded as a safer ASIT and can be administrated at home, SLIT has started to become the dominant usage of ASIT in Europe.

There is a however still a need to improve the effect of SLIT, in order to reduce the number of administered doses and also to be able to lower the allergen dose. Moreover, a safer and more effective SLIT is also needed in order to approach food allergy with SLIT.

Improving oral tolerance, by means of a more effective tolerogenic process, would lead to a more efficient SLIT and hence, allergen dosage and number of administrations could be reduced, giving both cost- and patient benefits. Further, it is envisaged that improved tolerogenic processing may imply that also strong allergens associated with dangerous side effects may be used in SLIT, as the dose required to induce tolerance may be lowered.

Although ASIT has clear benefits, it is not a widely used treatment principle for desensitizing allergic subjects. Inconvenience is one of the primary reasons for discontinuation of ASIT. In particular, the adherence to subcutaneous administered allergen in SIT treatment is problematic due to time constraints, adverse reactions and inconvenience. In WO 2010/146171 these inconveniences are targeted by directly administering the allergen slowly to subcutaneous tissue by subcutaneous infusion using an infusion pump. The oral administration route, e.g. SLIT, has also been used to overcome these inconveniences.

Present immunotherapy is also expensive. In SCIT the cost is mainly due to the tedious process of providing the allergen in a safe manner, which includes medical surveillance. The cost of SLIT relates more to the need of high doses of the allergen. Further, while the effect usually is maintained over time, it may diminish and repeated ASIT may thus be required to maintain adequate tolerance.

Thus improved ASIT, and especially SLIT, would be desirable. Preferably, an improved ASIT would result in the usage of lower allergen doses and fewer administrations occasions. The effect may hopefully also be prolonged effect, whereby the ASIT procedure would have to be repeated less often. Further, improved ASIT could hopefully reduce the risk for untoward reactions, such as anaphylaxis. The present invention thus aims to improve mucosal ASIT.

### SUMMARY

The present invention seeks to mitigate, alleviate, circumvent or eliminate at least one, such as one or more, of the above-identified deficiencies. Accordingly there is, according to one aspect of the invention, provided a superantigen for use in mucosal allergen specific immune therapy (ASIT) in a non-human mammal. The superantigen may be mucosally administered before, or with, the allergen to the non-human mammal. To facilitate co-administered, the superantigen and the allergen may be formulated into a single composition with at least one pharmaceutical acceptable carrier or excipient.

According to an aspect of the invention, the superantigen and/or the allergen is orally administered, e.g. sublingually.

According to an aspect of the invention, the administration of the superantigen and the allergen is repeated, the subsequent administration being performed at least 4 hours after the preceding administration but less than 2 weeks after the preceding administration.

According to an aspect of the invention, the non-human mammal to undergo ASIT, including the use of the superantigen, is at least 6 months old.

According to an aspect of the invention, the superantigen is selected from the group consisting of: SEA, SEB, SEC1, SEC2, SEC3, SED, SEE, SEG, SEH, SEI, SEJ, SEK, SEL, SEM, SEN, SEO, SEP, SER, SEQ, SER, SEU, SEV and TSST-1, or a mixture thereof.

According to an aspect of the invention, the allergen specific immune therapy (ASIT) targets allergies selected from the group consisting of canine atopic dermatitis (CAD) and food allergy. The allergen may be selected from the group consisting of environmental allergens (e.g. tree-, grass-, or wood-pollen, house dust mites, mold spores and fleas) and food allergens (e.g. beef protein, chicken protein, pork protein, corn protein, wheat protein soybean protein or egg protein).

According to another aspect of the invention, there is provided a composition comprising a superantigen, an allergen and at least one pharmaceutical acceptable carrier or excipient. The composition may be used in mucosal allergen specific immune therapy (ASIT) in a non-human mammal, as outlined above.

Further advantageous features of the invention are defined in the dependent claims. In addition, advantageous features of the invention are elaborated in embodiments disclosed herein.

### DETAILED DESCRIPTION

Several embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

In humans, the presence of a complex gut microbiota early in life is protective against allergy development in general (33-36). In the prospective IMMUNOFLORA birth cohort, it was observed that early colonization, i.e. within 4 weeks after birth, in the gut by S. aureus, but not other typical cultivable gut bacteria, was associated with protection from food allergy in the first 18 months of life (37). S. aureus produce a range of exotoxins which bind to MHC class II molecules on antigen-presenting cells and to the T cell receptor of a large portion of all T cells (38, 39). S. aureus enterotoxins strongly affect gut T lymphocytes, leading to activation of the gut nervous system (40), inducing vomiting and diarrhea, “food poisoning”.

It has previously been shown that S. aureus enterotoxins staphylococcal superantigens may be used to prophylactically prevent development of allergies in children (cf. WO 2006/009501), as well as in puppies (cf. WO 2013/119170) if administered early in life, i.e. within weeks after birth. This effect is however only present in newborns. As shown by the present inventors, the same mechanism is not functional in an adult mammals, e.g. mouse, having a mature immune system (41). Further, the mature immune system is very reactive to superantigens, which are the causative agent in certain types of food poisoning.

The present inventors have surprisingly found that exposure of adult mice with a mature immune system to staphylococcal superantigen shortly before oral administration of an allergen provides an enhanced tolerance to the allergen of concern. However, administration of staphylococcal superantigens alone does not have any effect on the induction of tolerance, confirming that the underlying mechanism is distinct from the one underlying the one seen when superantigens are administered within days after birth (47), i.e. neonatal administration of superantigens.

Without being bound by any theory, the finding may be explained by S. aureus enterotoxins, i.e. superantigens, activating gut-resident T cells, which, in turn, leads to an effect on the epithelial cells promoting their tolerogenic processing capacity. In accordance with this hypothesis increased density of intraepithelial CD8α⁺ T-cells after SEA-exposure in biopsies from the small intestine of donor mice was also found.

Further, it is known that gastrointestinal infections in early childhood are linked to protection from allergy development, while exposure to airway infections is much less effective (42). Activation of the gut epithelium promoting tolerogenic processing might be a mechanism involved in the improved tolerance to innocuous antigens (“allergens”) in young children growing up in unhygienic conditions.

The present inventors studied tolerogenic gut processing by transfer of serum from ovalbumin-fed donors to naïve recipient mice, followed by examination of ovalbumin-specific sensitization and Th2-driven airway-inflammation in the recipients. It has previously been shown that feeding a protein antigen leads to protection in the OVA-asthma model (37) as does transfer of serum from a fed donor (43).

Further, it was surprisingly found that serum from donors that were pretreated with S. aureus enterotoxins (SE) a few days prior to feeding rendered the recipients more tolerant than recipients of serum from mice that were not pretreated with S. aureus enterotoxin. Protection in the OVA-asthma model was observed as reduction of infiltrating eosinophils in BAL fluid. In addition, recipients of serum from donors that were SEA-treated prior to antigen administration, had significantly reduced production of IL-5 and IL-13 by lung cells stimulated by the recall antigen ovalbumin, compared to recipients of serum from donors that had not been treated with SEA prior to tolerogenic feeding of ovalbumin.

Thus, mice that received serum from SEA-treated ovalbumin fed donors were more effectively tolerized and developed a milder degree of allergic airway inflammation to the allergen that had been fed to the serum donors, compared to mice that received serum from ovalbumin fed non-SEA-treated donors.

An embodiment of the invention thus relates to the use of at least one superantigen in mucosal Allergen Specific Immune Therapy (ASIT) in a non-human mammal to enhance the effect thereof. Similarly, an embodiment relates to superantigen for use in mucosal ASIT in a non-human mammal to enhance the effect thereof. In such use, the superantigen is typically mucosally administered before, or with, the allergen to the non-human mammal. Further, yet another embodiment relates to a method of enhancing the effect of mucosal ASIT in a non-human mammal, wherein a superantigen is mucosally administered before, or with, the allergen to a subject suffering from hypersensitivity toward the allergen. Yet another embodiment relates to the use of at least one superantigen in the manufacture of a medicament for use in Allergen Specific Immune Therapy (ASIT) in a non-human mammal. As already described, ASIT is based upon administration of small amounts of a specific allergen, or a mixture of allergen(s), i.e. the agent(s) to which the non-human mammal is hypersensitive, to a non-human mammal in order to reduce the non-human mammals sensitivity towards the allergen(s). Thus, ASIT is only applied post the neonatal stage, most typically also post the infant stage, in subjects with a mature immune system.

As for normal ASIT, the age of the non-human mammal, when a superantigen is used in the present mucosal Allergen Specific Immune Therapy (ASIT), is typically at least 6 months, such as at least 1 year, or at least 2 years.

Mucosal ASIT aims to reduce the immune responses upon subsequent natural exposure to an allergen, by administering a low dose of the allergen in a controlled manner to a subject in need thereof. As the effect of the superantigen is to improve the tolergenic processing of the antigen, mucosal ASIT, as used herein, refers to administration routes wherein the allergen is processed into a tolergen subsequent to its administration. Such routes include various administration routes to mucous membranes, i.e. mucosal administration, but not various types of injections, such as subcutaneous injection. In administration onto a mucous membrane, the allergen may be administered onto the nasal mucous membrane, i.e. nasally, onto mucous membrane in the oral cavity, e.g. sublingually or buccally, or onto the intestinal mucous membrane, i.e. enterally.

According to an embodiment, the allergen is administered orally. Oral administration of allergens in mucosal ASIT, includes, as known to the skilled person, various types of enteral, buccal and sublingual administration, such as spit and swallow sublingual administration. The allergen may also be administered nasally or rectally. According to an embodiment, wherein the allergen is administered orally, the allergen is administered sublingually (corresponding to SLIT), such as by swallow sublingual administration.

Typically, the superantigen is administered in the same manner, i.e. by the same route, as the allergen, although it is not necessary. Various routes of administering superantigen to have effect on the immune system have been described in WO 2006/009501 and WO 2013/119170. According to an embodiment the superantigen is administered orally, such as sublingually, buccally, or enterally. According to an embodiment, wherein the superantigen is administered orally, the superantigen is administered sublingually, such as by swallow sublingual administration.

In use of superantigen(s) to enhance the effect of mucosal ASIT, the superantigen is administered shortly before, or along with the allergen. Further, as the effect of administrating the superantigen will abate, the time span between administration of the superantigen and the allergen should not be too long. Thus, the superantigen is typically administered less than 7 days, such as less than 6, 5, or 4 days, before administration of the allergen. Preferably, the superantigen is administered less than 18 hours before, such as less than 12, 8, 6, 4, 2 or 1, hour(s) before, the administration of the allergen.

According to an embodiment, the superantigen is co-administered with the allergen. Co-administration is deemed to be practical way of administering the superantigen and allergen. Thus, the superantigen and the allergen may be formulated into a single composition with at least one pharmaceutical acceptable carrier or excipient. As used herein pharmaceutical acceptable carrier or excipient refers to those carriers or excipients which are, within the scope of sound medical judgment, suitable for contact with the tissues of animals without excessive toxicity, irritation, allergic response, or other problem complications commensurate with a reasonable benefit/risk ratio.

Accordingly, a further embodiment relates to composition comprising a superantigen, an allergen, and at least one pharmaceutical acceptable carrier or excipient. Evidently, such a composition may be used in therapy in a non-human mammal, such as mucosal allergen specific immune therapy (ASIT) in a non-human mammal. Further, it may be used to manufacture a medicament for use in mucosal allergen specific immune therapy (ASIT) in a non-human mammal. A further embodiment relates to mucosal ASIT in a non-human mammal, wherein the composition is mucosally administered to a subject suffering from hypersensitivity toward the allergen

Further, it may be advantageous to repeat the administration of the allergen and/or the superantigen. The administration of the superantigen and/or the allergen may thus be repeated at least 2 times, preferably at least 3 times, such as at least 5, at least 7 or even at least 10 times. In repeating the administration, the subsequent administration(s) is preferably performed at least 4 to 12 hours after the preceding administration. Further, the subsequent administration(s) is preferably performed less than 2 weeks, such as less than 1 week, after the preceding administration.

According to an embodiment, the allergen and the superantigen are administered one, two or three times daily. Further, according to an embodiment, the allergen and the superantigen are administered at least every week, such as at least every second day or daily, for at least 1, such as at least 2, 3, 6, or 12 months.

The effect of improving the tolergen processing is believed to be due to the superantigens unique ability in affecting cells of the immune system; especially T lymphocytes. Thus, the effect is deemed to not be limited to a single or a few superantigens, but to be shared among in principle all superantigens.

Superantigens are mainly associated with Staphylococcus aureus and Streptococcus pyogenes, but also Streptococcus equi has been shown to produce superantigens (45)

According to an embodiment, the superantigen is a staphylococcal enterotoxin or a staphylococcal enterotoxin-like superantigen. The superantigen may thus be selected from the group consisting of: SEA, SEB, SEC1, SEC2, SEC3, SED, SEE, SEG, SEH, SEI, SEJ, SEK, SEL, SEM, SEN, SEO, SEP, SER, SEQ, SER, SEU, SEV and TSST-1.

Further, the superantigen may also be a streptococcal superantigen. This type of superantigen has the same mode of action as S. aureus superantigens. These superantigens presently include the following toxins from S. pyogenes: SPE-A, SPE-C, SPE-H, SPE-I, SPE-J, SPE-K/L, SPE-L/M, SPE-M, SSA, SMEZ-1, SMEZ-2, the following toxins from S. equi: SE-PE-H, SE-PE-I, SPE-L_{Se}, and SPE-M_{Se}, as well as the following toxins from S. dyslalactiae; SPE-G^{dys}, and SDM.

As discussed in by Lina et al in (46), not all staphylococcal enterotoxins have emetic properties. Although, the superantigen may be administered in a low does not necessarily being emetic, it may according to an embodiment be preferred to use a superantigen being less, or not all, emetic. Thus, the superantigen may according to such an embodiment be selected from the group consisting of: SEK, SEL, SEM, SEN, SEO, SEP, SEQ, and SEU. These superantigens have also been denoted SElK, SElL, SElM, SElN, SELO, SElP, SElQ, and SElU in the art. The letter “l” denotes that they are enterotoxin-like, i.e. that they do have superantigen properties, but that they may have less adverse effects.

Further, not only natural superantigens may be used to enhance the effect of mucosal ASIT, but also derivatives thereof, as long as they have superantigen activity. As superantigens are proteins, various ways of obtaining derivatives are known to the skilled person, such as amino acid substitution, deletion, or insertion as well as addition at the N-terminus or C-terminus of the protein. Substitution(s), insertion(s) and addition(s) may be performed with natural as well as non-natural amino acids. One type of derivatives of interest may be fragments of natural superantigens, i.e. proteins and peptides consisting of only part of the sequence of the full-length protein. Further, natural superantigens may be substituted with HIS-tags to facilitate purification, as well as PEG-moieties and other types of moieties affecting the solubility of the protein. According to an embodiment, superantigen, as used herein, relates to natural as well as unnatural superantigens, e.g. derivatives of natural superantigens. According to another embodiment, superantigen, as used herein, relates to natural superantigens.

Mucosal allergen specific immune therapy (ASIT) may target various allergic manifestations in non-human mammals, such as dogs, cats and horses. According to an embodiment, the non-human mammal that undergoes mucosal ASIT supplemented with a superantigen is a dog, a cat or a horse, such as a cat or a dog, e.g. a dog. The allergen may be of various types, such as environmental allergens (tree-, grass-, or wood-pollen, house dust mites, mold spores and fleas), food allergens (food proteins), e.g. beef protein, chicken protein, pork protein, corn protein, wheat protein soybean protein or egg protein. According to an embodiment, the allergen is from house dust mites.

In embodiments, wherein the non-human mammal is a dog, the dog may be a West Highland White Terrier, Boston Terrier, Boxer, Staffordshire Bullterrier or a French Bulldog. The dog may be suffering from food allergy, e.g. food induced cutaneous adverse reaction (FICAR), or canine atopic dermatitis.

Two of the most common allergic disorders in pet dogs are canine atopic dermatitis (CAD) and food allergies. Canine atopic dermatitis is a pruritic skin disease with typical location and appearance, i.e. affecting the face, ears, paws, extremities, and/or ventrum. Often, the dog also has IgE antibodies to environmental allergens, but this is not clearly linked to disease presentation, also known as sensibilization. Otitis externa and skin infections due to staphylococci and yeasts commonly accompany CAD, due to impaired skin barrier defense in this disease. The typical age of onset of CAD is reported to be between 6 months and 3 years.

CAD shares many features with human atopic dermatitis, such as similar histopathology, pruritus as the predominant clinical sign and impaired skin barrier function. No prophylactic or curative treatment is at hand for this disorder. Canine allergy is a complex, lifelong disease generally requiring lifelong treatment. However, immunotherapy via sublingual administration of the allergen (SLIT), have shown promising results in double blind study with atopic Beagles, sensitized to dust mites (DM, Dermatophagoides farinae), timothy grass (TG) and ragweed (RW), as reported by Marsella (27) and Marsella and Ahrens (poster entitled “Investigations on the effects of sublingual immunotherapy on clinical signs and immunological parameters using a canine model of atopic dermatitis: a double blinded, randomized, controlled study.”).

According to an embodiment, the mucosal ASIT, whose effect the superantigen is to increase, targets allergies selected from the group consisting of canine atopic dermatitis (CAD) and food allergies. Further, it may target allergic manifestations such as atopic dermatitis.

Similar, a composition comprising a superantigen, an allergen and at least one pharmaceutical acceptable carrier or excipient may be used in ASIT targeting allergies selected from the group consisting of canine atopic dermatitis (CAD) and food allergies. Further, it may target allergic manifestations, such as atopic dermatitis.

As the use of S. aureus enterotoxin will improve the allergen processing, the administrated amount of the allergen will be lower than in typical immunotherapy. By using a lower dose, side effects resulting from the intake of an allergen med be reduced and alleviated.

The use of superantigen in improving ASIT is not limited to single allergens. Thus, more than one type of allergen may be administered subsequent or along with the administration of the superantigen. The allergens may be administered together or one-by-one.

Typically the superantigen will be administered as part of a composition comprising at least one pharmaceutical acceptable carrier or excipient. The composition and contents of the composition will depend on the administration route. According to an embodiment the same type of formulation as used for allergen is used for the superantigen. Further, as already mentioned the superantigen and the allergen may be co-formulated into a single composition, whereby they may be co-administered.

Compositions comprising superantigen and optionally an allergen (if not to be administered separately) for use in mucosal ASIT may, for example, be in the form of tablets, pills sachets, vials, hard or soft capsules, aqueous or oily suspensions, aqueous or oily solutions, emulsions, powders, granules, syrups, elixirs, lozenges, reconstitutable powders, liquid preparations, sprays, creams, salves, jellies, gels, pastes, ointments, liquid aerosols, dry powder formulations, or HFA aerosols.

A composition comprising a superantigen and optionally an allergen (if not to be administered separately) for use in mucosal ASIT, may be in a form suitable for administration through oral routes, e.g. enteral, buccal, or sublingual. Further, but less preferred it may be for administration by inhalation or insufflation (e.g. nasal, tracheal, bronchial) routes. According to an embodiment, the composition is for sublingual administration, such as sublingual swallow administration. A composition for sublingual administration may comprise the superantigen as well as the allergen.

Depending upon the type of hypersensitivity, the allergy, and subject to be treated as well as the route of administration, the compositions may be administered at varying doses. A suggested dose concentration of administration of a solution or a suspension of bacterial superantigen(s) is 10 to 100 μg/ml, such as about 40 μg/ml. The dose of the superantigen(s) is according to an embodiment in the range 1 to 750 μg per kg bodyweight, such as 15 to 300 μg per kg bodyweight, or 30 to 180 μg per kg bodyweight.

For oral, e.g. enteral, buccal or sublingual, administration, the bacterial superantigen may be combined with various excipients. Solid pharmaceutical composition for oral, e.g. enteral buccal, or sublingual, administration often include binding agents (for example syrups and sugars, acacia, gelatin, sorbitol, tragacanth, polyvinylpyrrolidone, sodium lauryl sulphate, pregelatinized maize starch, hydroxypropyl methylcellulose, lactose, starches, modified starches, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone and sodium alginate), disintegrants (such as starch and preferably corn, potato or tapioca starch, alginic acid and certain complex silicates, polyvinylpyrrolidone, sucrose, gelatin, acacia, sodium starch glycollate, microcrystalline cellulose, crosscarmellose sodium, crospovidone, hydroxypropyl methylcellulose and hydroxypropyl cellulose), lubricating agents (such as magnesium stearate, sodium lauryl sulfate, talc, silica polyethylene glycol waxes, stearic acid, palmitic acid, calcium stearate, carnuba wax, hydrogenated vegetable oils, mineral oils, polyethylene glycols and sodium stearyl fumarate) and fillers (including high molecular weight polyethylene glycols, lactose, sugar, calcium phosphate, sorbitol, glycine magnesium stearate, starch, glucose, lactose, sucrose, rice flour, chalk, gelatin, microcrystalline cellulose, calcium sulphate, xylitol and lactitol). Such compositions may also include preservative agents and anti-oxidants.

Liquid pharmaceutical compositions for oral, e.g. enteral, buccal, or sublingual, administration may be in the form of, for example, solutions, dispersions, emulsions, syrups, or elixirs, or may be presented as a dry product for reconstitution with water or other suitable vehicle before use. Such liquid compositions may contain conventional additives such as suspending agents (e.g. sorbitol, syrup, methyl cellulose, hydrogenated edible fats, gelatin, hydroxyalkylcelluloses, carboxymethylcellulose, aluminium stearate gel, hydrogenated edible fats) emulsifying agents (e.g. lecithin, sorbitan monooleate, or acacia), aqueous or non-aqueous vehicles (including edible oils, e.g. almond oil, fractionated coconut oil) oily esters (for example esters of glycerine, propylene glycol, polyethylene glycol or ethyl alcohol), glycerine, water or normal saline; preservatives (e.g. methyl or propyl p-hydroxybenzoate or sorbic acid) and conventional flavoring, preservative, sweetening or coloring agents. Diluents such as water, ethanol, propylene glycol, glycerin and combinations thereof may also be included.

Other suitable fillers, binders, disintegrants, lubricants and additional excipients are well known to a person skilled in the art.

Oral delivery of therapeutic agents in general is a preferred mode of administration due to its convenience and simplicity, both contributing to better patient compliance. Recombinant technology has made available a wider selection of proteins and polypeptides for use as therapeutic agents, and oral delivery of proteins and polypeptides is thus of increasing interest and value. However, because proteins and polypeptides can be unstable during storage, leading to loss of biological activity, an oral formulation is preferably designed to optimize stability for retention of activity during storage and upon administration. According to an embodiment, a composition comprising a bacterial superantigen optionally an allergen (if not to be delivered separately) for use in mucosal ASIT is administered orally.

Formulation factors that require consideration of design of an oral formulation of a protein or polypeptide, such as superantigen and/or an allergen, include the solution behavior of the protein or polypeptide in aqueous and non-aqueous solvents and the effect of ionic strength, solution pH, and solvent type on the stability and structure of the protein or polypeptide. The effect of temperature during formulation on the stability and structure of the protein or polypeptide must also be considered, as should the overall suitability of the formulation for incorporation into an oral dosage form, and particularly into an oral liquid dosage form, such as a gelatin capsule or syrup.

For nasal administration or administration by inhalation, the superantigen may be delivered in the form of a solution, dry powder or suspension. Administration may take place via a pump spray container that is squeezed or pumped by the administrator or through an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. The bacterial superantigen may also be administered via a dry powder inhaler, either as a finely divided powder in combination with a carrier substance (e.g. a saccharide) or as microspheres. The inhaler, pump spray or aerosol spray may be single or multi dose. The dosage may be controlled through a valve which delivers a measured amount of active compound.

Various compositions of allergen(s) for mucosal ASIT are known in the art. As an example, the allergen may be formulated for sublingual swallow administration.

In some embodiments, the allergen(s) are formulated as microspheres. This may be achieved by dispersing the allergen(s) in an aqueous solution. The solution is then sprayed onto a core particle resulting in the formation of a microsphere. The allergen coating may constitute 1-10 wt. % of the microsphere. Examples of suitable core particles include nonpareils composed of sugar and/or starch. In order to protect the allergen upon passage through the stomach, the microspheres may be coated. They may be coated with a polymer in solution which solidifies to become acid resistant coating. A non-limiting example of the solution is a water based emulsion of the polymer. Once the allergen has passed through the stomach, it reaches the small intestines wherein it is to be processed and taken up as a tolerogen. As already described, the allergen may be co-formulated with the superantigen.

The coating material may further include a plasticizer, such as triethylcitrate, to improve the continuity of the coating. While plasticizers can be liquid, they are distinct from solvents as they remain within the coating material to alter its physical characteristics. Plasticizers do thus not act to dissolve the allergen. The coating may further include talc to prevent sticking between the microsphere particles and/or an antifoaming agent, such as sorbitan sesquioleate) or silicone.

According to an embodiment the allergen is used or formulated in combination with a stabilizing agent. The stabilizing agent may provide physical protection for the allergen. Non-limiting examples of stabilizing agents include therapeutically inactive water soluble sugars such as lactose, mannitol and trehalose. Further. polyvinylpyrrolidone may be used to aid the binding of allergen to a nonpareil.

Without further elaboration, it is believed that one skilled in the art may, using the preceding description, utilize the present invention to its fullest extent. The above preferred specific embodiments are, therefore, to be construed as merely illustrative and not limitative to the disclosure in any way whatsoever.

Although the present invention has been described above with reference to (a) specific embodiment(s), it is not intended to be limited to the specific form set forth herein. Rather, the invention is limited only by the accompanying claims and, other embodiments than the specific above are equally possible within the scope of these appended claims, e.g. different than those described above.

In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous.

In addition, singular references do not exclude a plurality. The terms “a”, “an”, “first”, “second” etc do not preclude a plurality.

### BRIEF DESCRIPTION OF DRAWINGS

These and other aspects, features and advantages of which the invention is capable of will be apparent and elucidated from the following description of embodiments of the present invention, reference being made to the accompanying drawings, in which

FIG. 1 depicts over view of the experimental protocol used for evaluating the ability of S. aureus enterotoxin A (SEA) to enhance the tolerogenic processing of ovalbumin (OVA).

FIG. 2 Cells in bronchoalveolar lavage fluid (BALf) of recipient mice. Bronchoalveolar lavage was performed after sensitization and challenge of recipient mice (see FIG. 1). Infiltrating cells were counted and stained by May-Grünwald Giemsa to distinguish the eosinophils. (A) Concentration of infiltrating cells/ml in BAL fluid. (B) Fraction of eosinophils among infiltrating cells. (C) Concentration of eosinophils/ml.

FIG. 3 Cytokines in supernatants from in vitro stimulated lung cells. Cells prepared from lung tissue, collected from recipient mice after sensitization and challenge (see FIG. 1), were re-stimulated with ovalbumin in vitro for 48 h. Levels of IL-5 and IL-13 in the culture medium were measured by ELISA. (A) IL-5 (pg/ml) (B) IL-13 (pg/ml).

FIG. 4A Number of CD8α-positive cells in biopsies from SEA-exposed and control mice (CD8 α⁺ cells/mm²)

FIG. 4B Relative MHC class II-staining of biopsies from SEA-exposed and control mice (% of the epithelium stained positive).

### EXAMPLE NO. 1

Methods

Experimental Protocol

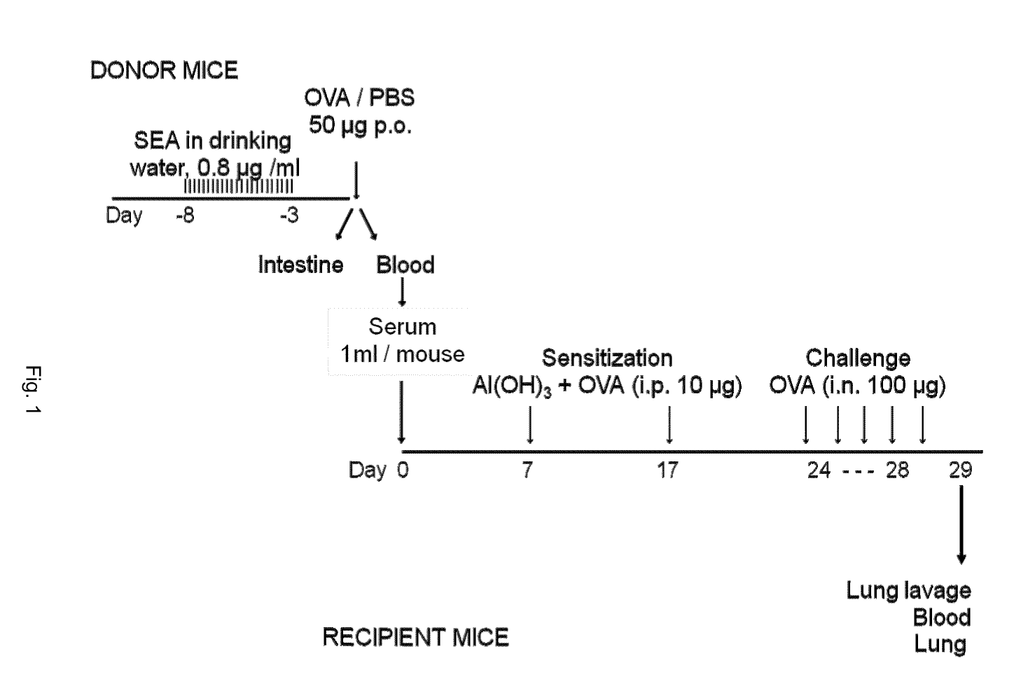

An overview of the experimental protocol is depicted in FIG. 1. In short, donor mice were exposed to S. aureus enterotoxin A (SEA) in the drinking water (daily dose: 4 μg/mouse) for 5 days; controls were given standard drinking water. After a resting phase of 3 days, when all mice received standard drinking water, the mice were starved overnight and fed 50 μg ovalbumin (OVA) in PBS by gavage, or sham treated with PBS only. One hour after feeding, donor mice were sacrificed and bled by cardiac puncture. At this time point, sections of small intestines of the donor mice were prepared, stained and examined (n=8). Serum was transferred into naïve recipient mice (1 ml serum/recipient) by i.p injection (n=16). Recipient mice were tested for reactivity in a model of allergic airway inflammation. The recipient mice were immunized with alum-adsorbed OVA i.p. (10 μg) twice and challenged with repeated intranasal instillation of OVA for 5 consecutive days. The day after the last challenge, the mice were sacrificed and subjected to bronchoalveolar lavage. Lung tissue and blood was also collected for analysis.

Animals

BALB/c mice (B & K, Stockholm, Sweden) were housed under specific pathogen-free conditions in the animal facilities of the Medical Faculty of the University of Gothenburg. The experiments were performed with the permission of the Ethics Committee, University of Gothenburg.

S. aureus Enterotoxin-Exposure and Adoptive Serum Transfer

For a schematic overview of the protocol, see FIG. 1. Donor mice (6-8 weeks old males) were given drinking water with or without (control mice) 0.8 μg/ml S. aureus enterotoxin A (SEA; Sigma Chemical Co., St. Louis, Mo.) for five days. A mouse drinks about 5 ml of water daily, corresponding to 4 μg SEA. Three days later, they were starved overnight and then fed 0.3 ml phosphate-buffered saline (PBS) with or without 50 mg ovalbumin (OVA; grade V, Sigma). One hour later, the mice were anaesthetized (Isoflurane, Baxter Medical, Kista, Sweden) and bled by cardiac puncture. Blood from each group of mice (SEA-PBS, SEA-OVA, control-PBS or control-OVA, respectively) was pooled, allowed to clot and centrifuged twice at 3,000×g for 10 min. 1 ml serum was injected intraperitoneally (i.p.) into naïve BALB/c recipient mice, matched for sex and age.

The Ovalbumin-Asthma Model

Recipient mice were tested for tolerance in a model of ovalbumin-induced allergic airway inflammation the OVA-asthma model. Feeding of OVA is known to reduce airway inflammation in this model, i.e. oral tolerance is induced to the model allergen(37-39). Recipients were sensitized by two i.p. injections of 10 μg ovalbumin (grade V, Sigma), dissolved in 50 μl PBS and mixed with 100 μl of aluminium hydroxide gel (Sigma). Sensitization was performed 7 and 17 days after transfer of serum from fed mice (see FIG. 1). Allergic airway inflammation was elicited by repeated intranasal challenge with ovalbumin. Thus, 100 μg ovalbumin in 25 μl PBS was administered daily on 5 consecutive days to briefly anesthetized mice (Isoflurane, Baxter Medical, Kista, Sweden). The first challenge dose was given day 24 after serum transfer, i.e. 6 days after the second sensitizing i.p. dose of ovalbumin.

Twenty-four hours after the last challenge dose, recipient mice were anesthetized with xylazine (130 mg/kg, Rompun; Bayer, Leverkusen, Germany) and ketamine (670 mg/kg, Ketalar; Pfizer AB, Täby, Sweden). Blood was obtained by cardiac puncture for determination of total and ovalbumin-specific IgE. Lung lavage was performed to enumerate infiltrating eosinophils. Lung tissue was collected for in vitro restimulation of lung resident immune cells with ovalbumin and determination of cytokine production in response to this antigen stimulation (see below).

Bronchoalveolar Lavage.

Lung lavage was performed to enumerate infiltrating eosinophilic polymophonuclear granulocytes (“eosinophils”). PBS (0.4 ml) was instilled twice through a tracheal cannula, followed by gentle aspiration. Cells were counted in a Haemocytometer (Bürker chamber). Aliquots of BAL fluid containing 10⁵ cells were cytocentrifuged (Shandon Southern, Runcorn, UK). After staining with May-Giemsa, the proportion of eosinophils was determined among 300 cells examined in high-powered microscopic fields. Counting of bronchoalveolar lavage cells and the proportion of eosinophils were performed by an investigator blinded regarding to the treatment given to the mice.

Ovalbumin-Induced Cytokine Production.

After lavage, one lung was excised and cut into pieces. Single-cell suspensions were obtained after digestion with collagenase (1 mg/ml; Sigma) and DNase (0.1 mg/ml; Sigma) for 20 min at 37° C. in Iscove's medium, followed by squeezing through a nylon filter. The cells were washed in medium and red blood cells were lysed with NH₄Cl (5 min, 37° C.). After washing, 5×10⁵ cells/well were seeded in 96-well U-bottomed plates (Nunc, Roskilde, Denmark) in Iscove's medium supplemented with 10% heat-inactivated fetal calf serum, 2 mM L-glutamine, 50 μg/ml gentamycin, and 50 μm 2-mercaptoethanol (all from Sigma). The cultures were stimulated with 500 μg/ml ovalbumin, or medium alone (blank). Supernatants were collected after 48 h and stored at −20° C. until analyzed. The levels of IL-10, IL-5, IL-13 and IFN-γ in supernatants were determined by sandwich enzyme-linked immunosorbent assay (ELISA) (R&D Systems detection kit), performed as follows: Costar plates were coated overnight at room temperature with capture antibody, washed ×3 with PBS and blocked for 1 h with PBS containing 1% BSA. Cytokine standards or sample (diluted 1:2, 1:10 and 1:50) were added and incubated for 2 h at room temperature. After washing ×3 with PBS with 0.05% Tween, detection antibody, diluted in PBS with 1% BSA, was added and incubated for 1 h. The plates were washed and incubated with streptavidin-horseradish peroxidase for 30 min and tetramethylbenzidine (TMB) liquid substrate (Sigma) for 20 minutes in the dark. The reaction was stopped with 1 M H₂SO₄ and the absorption at 450 nm was determined spectrophotometrically (Emax, Molecular Devices, Sunnyvale, Calif.). The detection limits were 70 pg/ml (IL-5) and 200 pg/ml (IL-13).

Determination of Ovalbumin-Specific IgE in Serum.

Ovalbumin-specific IgE antibodies were assayed by passive cutaneous anaphylaxis (PCA). Sprague-Dawley rats were anaesthetized (isoflurane inhalation followed by 8 mg/kg xylazine and 40 mg/kg ketamine i.p.). Mouse sera were diluted in twofold steps and 50 μl was injected intradermally into shaved dorsal skin of the rat. After 72 h, the rats were given 5 mg of ovalbumin in 1 ml PBS with 1% Evans' blue (Sigma) as an intravenous injection. They were sacrificed 1 h later. In a positive reaction, anti-ovalbumin antibodies of the IgE isotype are absorbed by Fc-epsilon receptors on tissue-bound mast cells. When ovalbumin is injected, mast cell-bound specific IgE reacts with the antigen, which activates release of histamine and leakage of dye-protein complexes into the tissue, leading to the appearance of a blue spot in the skin. The IgE anti-ovalbumin titer was defined as the reciprocal of the highest dilution giving a blue spot with a diameter of >2 mm.

Quantification of Total Serum IgE.

Total IgE concentrations in serum of recipient animals were determined by ELISA. Costar plates were coated with mouse anti-IgE (1 μg/ml; BD Biosciences Pharmingen), washed ×3 with PBS and blocked for 1 h with 1% bovine serum albumin (BSA). 50 μl of samples diluted 1:3-1:81 were added to the coated wells and the plates were incubated for 2 h at room temperature. After washing off non-bound serum constituents, biotinylated anti-mouse IgE (2 μg/ml; BD Biosciences Pharmingen) was added, followed by 1 h of incubation. The plates were washed and incubated with streptavidin-horseradish peroxidase and substrate was added, as described above (see: Ovalbumin induced cytokine production). The limit of detection was 5 ng/ml.

Examination of Intraepithelial Lymphocytes and MHC II Expression in Donor's Small Intestines

Mid-jejunal biopsies were excised from donor mice at the time for transfer, three days after the last SEA-treatment. Pieces of small intestines were placed in specimen moulds (Tissue-Tek Cryomould Biopsy; Miles Inc., Elkhart, Ind.) with Tissue-Tek O.C.T. compound (Sakura Finetek Europe BV, Zoeterwoude, the Netherlands), frozen instantly in isopentane cooled by liquid nitrogen, and stored at −70° C. Cryostat sections (6 μm thick) were prepared and fixed in cold acetone 50% for 30 s and 100% for 5 min. Endogenous peroxidase activity was blocked by incubation for 10 min in 1 U/l glucose oxidase (Type V-S; Sigma), 10 mM glucose and 1 mM NaN₃. Sections were incubated overnight at 4° C. with biotinylated monoclonals against I-A^{d} MHC class II or CD8a (both Pharmingen, San Diego, Calif.), in PBS with 0.1% saponine, followed by avidin-conjugated peroxidase (Vectastain ABC; Vector laboratories, Burlingame, Calif.) for 30 min and amino-ethyl-carbazole. The tissue was counter-stained with Mayer's haematoxylin and examined in a Leica Q500MC microscope using Leica Qwin Software by a group-blinded investigator (Leica, Cambridge, UK). MHC class II staining of epithelium was expressed as relative stained area (%) and intraepithelial lymphocytes as CD8α⁺ cells/mm² villus area. For both markers, 3 sections were analyzed from 8 each of mice per group.

Statistical Analysis

Kruskal-Wallis test was used to confirm significant differences between groups, followed by the Mann-Whitney U-test using Prism (GraphPad Software, San Diego, Calif.).

Results

Feeding of a dietary protein results in appearance of a tolerogenic form of the fed antigen in serum. The presence of such tolerogenic antigen can be demonstrated by transfer of serum to naïve recipients which will become actively tolerant to the antigen in question. To investigate the effect of S. aureus enterotoxin on tolerogenic processing, donor mice were exposed to SEA in the drinking water for 5 days, rested for 3 days and fed a tolerizing dose of ovalbumin. Serum collected shortly after feeding was transferred to naïve recipients, which were sensitized and challenged with ovalbumin in a model of Th2-mediated allergic airway inflammation. Tolerance was evaluated as reduction in infiltration of inflammatory cells into the lungs and reduction of ovalbumin-induced cytokine production by the cells extracted from the lung parenchyme. The experimental set-up is shown in FIG. 1 and described below.

Mice (6-8 weeks old) were given Staphylococcal enterotoxin A (SEA) in the drinking water (0.8 mg/ml) for 5 days. SEA was removed and the mice were left to rest for three days. Thereafter, mice (both SEA exposed and untreated SHAM controls) were fed by gavage either with ovalbumin (OVA; 50 mg) or with PBS (controls). The mice were sacrificed at 1 hour after feeding and blood was collected. Serum was prepared and injected intraperitoneally (i.p) (1 ml) into naïve recipient mice. At seven days after injection with serum all mice were introduced into an airway allergy model.

Reduced Eosinophil Infiltration in BAL after Transfer of Serum from SEA-Pretreated Donors

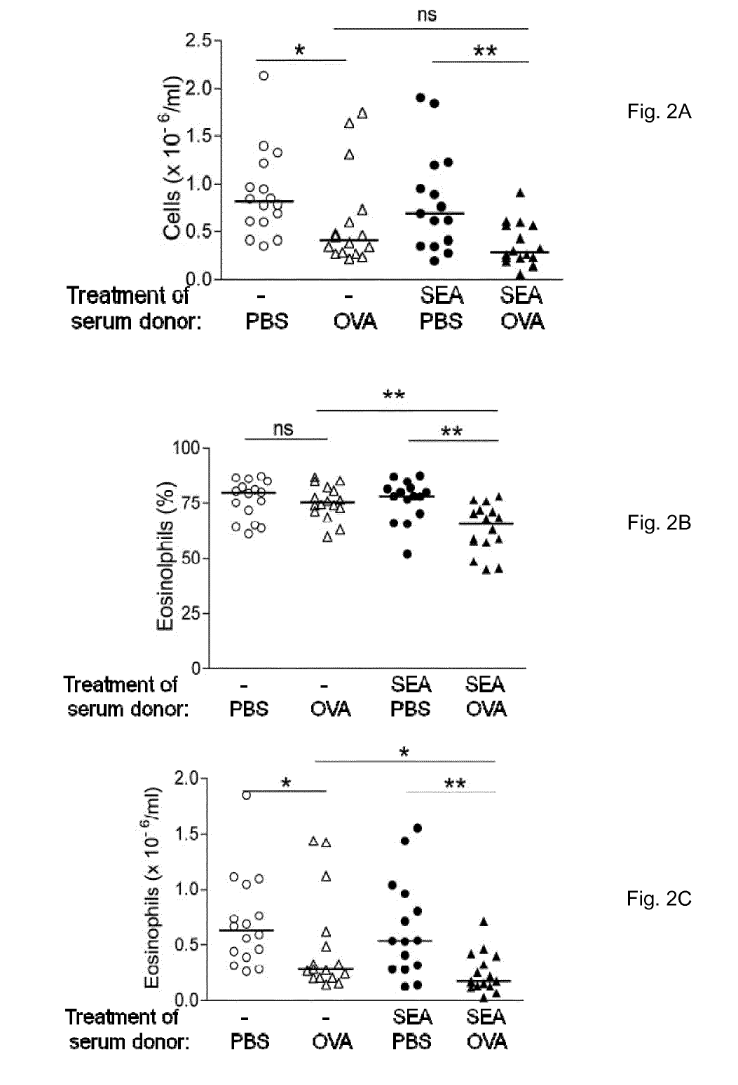

FIG. 2A shows the number of cells in the bronchoalveolar lavage (BAL) fluid in recipient mice sensitized and challenged with ovalbumin. To reveal the effect of SEA pretreatment on tolerogenic processing, we compared ovalbumin-specific tolerance in SEA-pretreated (SEA-OVA and SEA-PBS) and sham-treated (Ctrl-OVA and Ctrl-PBS) mice. Mice that had received serum from ovalbumin-fed donors had significantly fewer cells in the lavage fluid than mice that received serum from sham-fed donors. This was true whether the donors had been exposed to SEA 3 days prior to ovalbumin feeding (black symbols) or not (open symbols). This is in line with recent data from our group, showing that serum-transfer from ovalbumin-fed donors renders naive recipient mice tolerant to subsequent challenge with ovalbumin (44).

After sensitization and challenge with ovalbumin, the majority of cells in BAL fluid were eosinophils in all groups (FIG. 2B). In mice which had received serum from donor mice exposed to SEA prior to ovalbumin-feeding, the fraction of eosinophils was significantly reduced compared to SEA-pretreated non-fed mice. In contrast, the proportion of eosinophils was not significantly reduced in BAL fluid from mice that had received serum from ovalbumin-fed donors with no prior SEA-exposure. As a result, the fraction of eosinophils was significantly lower in BAL fluid of recipients of SEA-pretreated ovalbumin-fed mice, as compared to sham-treated ovalbumin-fed mice. The total number of infiltrating eosinophils, based on numbers of infiltrating cells and the fraction of eosinophils was correspondingly reduced (FIG. 2C). Recipients of serum from ovalbumin-fed mice had lower numbers of infiltrating eosinophils than recipients of serum from sham-fed mice, but the tolerance was more pronounced if the donors had been treated with SEA before feeding. Thus, the number of infiltrating eosinophils was significantly lower in recipients of serum from SEA-pretreated, ovalbumin-fed donors, than in recipients from sham-pretreated ovalbumin-fed donors (FIG. 2C). Of note, SEA treatment in itself did not significantly reduce cell infiltration or eosinophil proportion (Ctrl-PBS vs. SEA-PBS). Thus, the effect was antigen-specific and could not be due to a general effect of SEA pretreatment on e.g. inflammatory effector cells.

Decreased Production of IL-5 and IL-13 by Lung Cells after Transfer of Serum from SEA-Pretreated and OVA Fed Donors

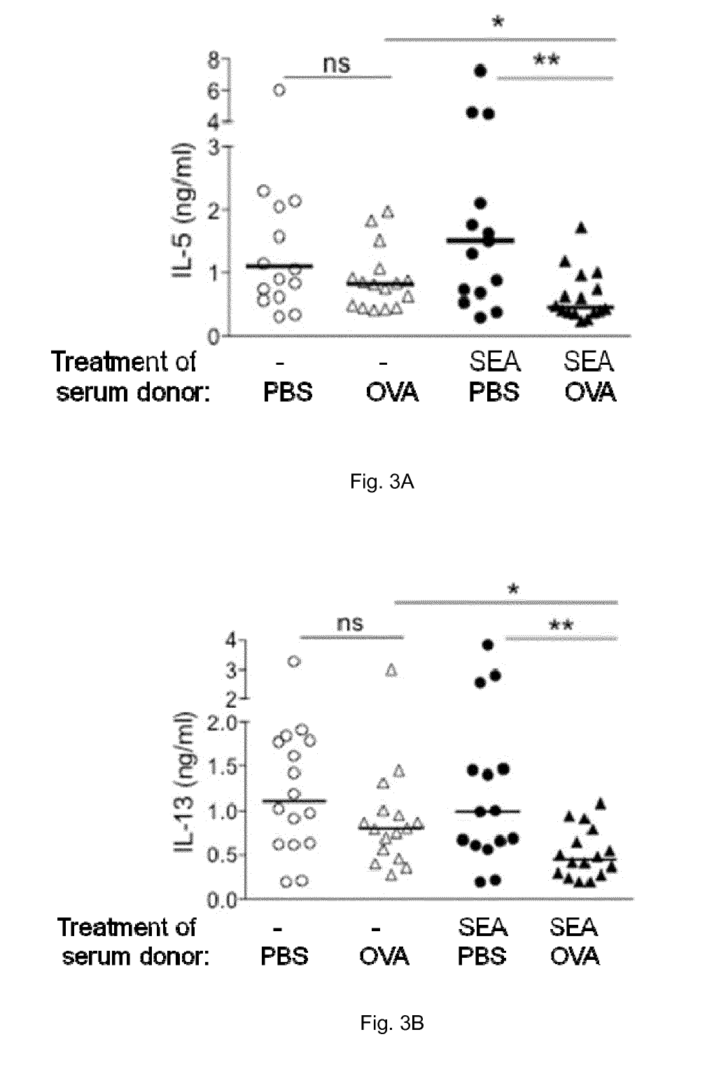

Single cell suspensions, prepared from lung tissue of recipient mice, were re-stimulated in vitro with ovalbumin and the cytokine production in response to this recall antigen was measured. With no prior SEA-treatment of the donors, production of IL-5 (FIG. 3A) and IL-13 (FIG. 3B) did not differ significantly between lung cells of recipients of serum from ovalbumin-fed and sham-fed donors. When donors were treated with SEA prior to ovalbumin-feeding, recipients of their serum showed significantly reduced lung cell IL-5 and IL-13 production compared to recipients of serum from sham-fed donors and ovalbumin-fed donors with no prior SEA-treatment.

As noted above, the effect was antigen dependent and not due to a general effect of SEA, since SEA pretreatment of the serum donors in itself did not reduce Th2 cytokine production (SEA-PBS vs. Ctrl-PBS) in the recipients. The levels of IL-10 did not differ between groups, and there were no detectable levels of IFN-γ in the cell culture supernatants. The serum IgE-levels did not differ between the groups (data not shown).

Increased Density of CD8α⁺ Intestinal Epithelial Lymphocytes in Small Intestinal Villi of SEA-Exposed Donor Mice

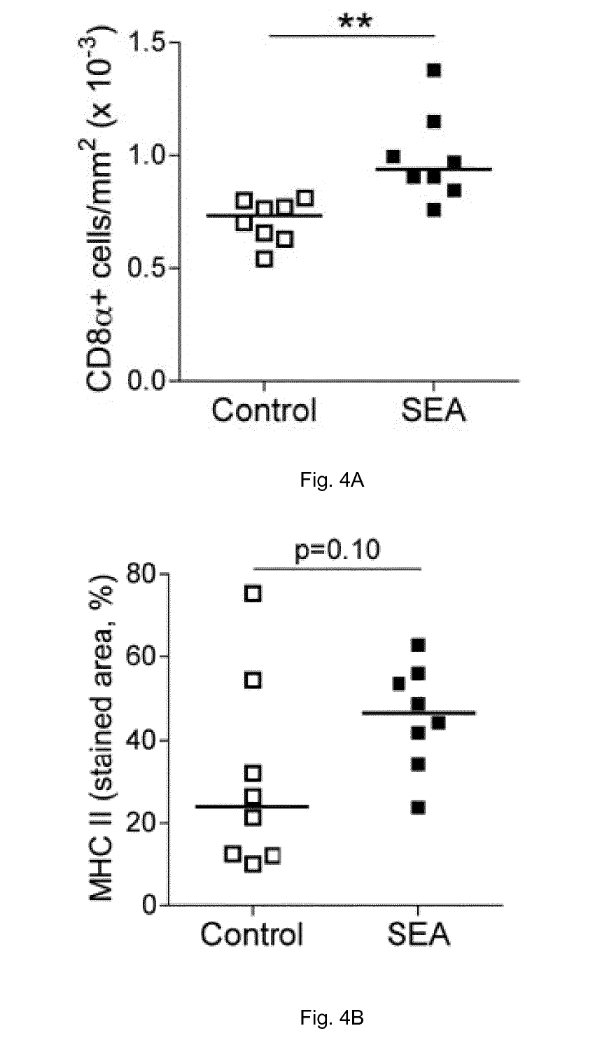

Small intestinal biopsies were obtained from SEA-treated and control donors at the time of serum transfer three days after the last SEA-exposure. Donor mice exposed to SEA had significantly increased density of CD8α⁺ intra-epithelial lymphocytes in the small intestine (FIG. 4A). The intestinal epithelial cells clearly tended to express more MHC class II in SEA treated group, p=0.10 (FIG. 4B).

### EXAMPLE NO. 2

Further, it was investigated whether sublingual immunotherapy (SLIT) treatment is effective in a mouse model of airway sensitization and whether administration of superantigen, staphylococcal enterotoxin A (SEA), together with the model antigen ovalbumin (OVA) has any an additional effect.

In short, female BALB/c mice, 7-8 weeks old, i.e. post the neonatal stage, were given SLIT treatment by sublingual administration of 100 μg OVA solution alone or together with SEA in various concentrations (0.38, 0.75, 1.5, and 3 μg, respectively). This treatment was given 10 times during two weeks. SLIT treated mice were then sensitized by intraperitoneal injections of alum-adsorbed OVA and subsequently challenged intranasally and analyzed for antibody levels, eosinophilia and cellular response.

The cellular response was evaluated as IFN-γ secretion from in vitro stimulated spleen cells, 2×10⁵ splenocytes were incubated at 37° C. together with OVA (0.5 mg/mL) and after three days of culture, supernatant was collected and analyzed for IFN-g by ELISA.

Preliminary data show that IFN-γ secretion from in vitro stimulated spleen cells were lower in mice given SEA together with OVA, in a dose-dependent matter, compared to mice given OVA alone. These results confirm that administration of a superantigen in conjunction to existing SLIT treatments has a beneficial effect, improving the efficiency of the SLIT treatment.

### REFERENCES

1. Berns, S. H., et al., Food allergy as a risk factor for asthma morbidity in adults. J Asthma, 2007. 44(5): p. 377-81.
2. Lund, E M et al., Health status and population characteristics of dogs and cats examined at private veterinary practice in the United States. J. Am. Vet Med. Assoc., 1999. 214 (1999): p. 1336-1341.
3. Griffin, C. E., DeBoer, D. J., The ACVD task force on canine atopic dermatitis (XIV): clinical manifestations of canine atopic dermatitis. Vet Immunol Immunopathol. 2001 Sep. 20; 81(3-4):255-69.
4. Agria insurance data, Updated Dog breed statistics: 2006-2011, www.agria.se
5. G. H. Nesbitt, G. S. Kedan, P. Caciolo, Canine atopy, part I. Etiology and diagnosis Comp. Cont. Educ. Pract. Vet., 6 (1984), pp. 73-84
6. Griffin, C. E., 1993. Canine atopic disease. In: Griffin, C. E., Kwochka, K., MacDonald, J. (Eds.), Current Veterinary Dermatology, The Science and Art of Therapy. Mosby Year Book, St. Louis, pp. 99-120.
7. M. N. Saridomichelakis, A. F. Koutinas, D. Gioulekas, L. Leontidis, Canine atopic dermatitis in Greece: clinical observations and the prevalence of positive intradermal test reactions in 91 spontaneous cases, Vet. Immunol. Immunopathol., 69 (1999), pp. 61-73
8. Husby, S., et al., Oral tolerance in humans. T cell but not B cell tolerance after antigen feeding. J Immunol, 1994. 152(9): p. 4663-70
9. Garside, P. and A. M. Mowat, Mechanisms of oral tolerance. Crit Rev Immunol, 1997. 17(2): p. 119-37.
10. Moreau, M. C. and G. Corthier, Effect of the gastrointestinal microflora on induction and maintenance of oral tolerance to ovalbumin in C3H/HeJ mice. Infect Immun, 1988. 56(10): p. 2766-8.
11. Bruce, M. G. and A. Ferguson, Oral tolerance to ovalbumin in mice: studies of chemically modified and ‘biologically filtered’ antigen. Immunology, 1986. 57(4): p. 627-30.
12. Strobel, S., et al., Immunological responses to fed protein antigens in mice. II. Oral tolerance for CMI is due to activation of cyclophosphamide-sensitive cells by gut-processed antigen. Immunology, 1983. 49(3): p. 451-6.
13. Karlsson, M., et al., “Tolerosomes” are produced by intestinal epithelial cells. Eur J Immunol, 2001. 31(10): p. 2892-900.
14. Jones S M et al. Clinical efficacy and immune regulation with peanut oral immunotherapy. J Allergy Clin Immunol 2009; 124:292-300, e1-97.
15. Longo G et al. Specific oral tolerance induction in children with very severe cow's milk-induced reactions. J Allergy Clin Immunol 2008; 121:343-7.
16. Meglio P et al. A protocol for oral desensitization in children with IgE-mediated cow's milk allergy. Allergy 2004; 59:980-7.
17. Patriarca G et al. Oral desensitizing treatment in food allergy: clinical and immunological results. Aliment Pharmacol Ther 2003; 17:459-65.
18. Staden U et al., Specific oral tolerance induction in food allergy in children: efficacy and clinical patterns of reaction. Allergy 2007; 62:1261-9.
19. Buchanan A D et al. Egg oral immunotherapy in nonanaphylactic children with egg allergy. J Allergy Clin Immunol 2007; 119:199-205.
20. Skripak J M, et al. A randomized, double-blind, placebo-controlled study of milk oral immunotherapy for cow's milk allergy. J Allergy Clin Immunol 2008; 122:1154-60.
21. Varshney P et al. A randomized controlled study of peanut oral immunotherapy: clinical desensitization and modulation of the allergic response. J Allergy Clin Immunol 2011; 127:654-60.
22. Keet C A, et al. The safety and efficacy of sublingual and oral immunotherapy for milk allergy. J Allergy Clin Immunol 2012; 129:448-55, e1-5.
23. Burks A W et al. Oral immunotherapy for treatment of egg allergy in children. N Engl J Med 2012; 367:233-43.
24. Enrique E et al. Sublingual immunotherapy for hazelnut food allergy: a randomized, double-blind, placebo-controlled study with a standardized hazelnut extract. J Allergy Clin Immunol 2005; 116:1073-9.
25. Kim E H et al. Sublingual immunotherapy for peanut allergy: clinical and immunologic evidence of desensitization. J Allergy Clin Immunol 2011; 127:640-6.e1.
26. DeBoer, D. J., Verbrugge, M., Morris, M., Changes in mite-specific IgE and IgG levels during sublingual immunotherapy (SLIT) in dust mite-sensitive dogs with atopic dermatitis, Proceedings of 24th Annual Congress of the ECVD-ESVD, 23-25 Sep. 2010, Firenze, Italy, Journal of Veterinary Dermatology, 21; 2010 page 531-2.
27. Marsella R., Tolerability and clinical efficacy of oral immunotherapy with house dust mites in a model of canine atopic dermatitis: a pilot study. Vet Dermatol. 2010 December; 21(6):566-71. doi: 10.1111/j.1365-3164.2010.00890.x.
28. Cox L, et al. Subcutaneous allergen immunotherapy for allergic disease: examining efficacy, safety and cost-effectiveness of current and novel formulations. Immunotherapy 2012; 4:601-16.
29. Calderon M A et al., Allergen-specific immunotherapy for respiratory allergies: from meta-analysis to registration and beyond. J Allergy Clin Immunol 2011; 127:30-8.
30. Compalati E, et al., Evidences of efficacy of allergen immunotherapy in atopic dermatitis: an updated review. Curr Opin Allergy Clin Immunol 2012; 12:427-33.
31. Canonica G W, et al. Sub-lingual immunotherapy: World Allergy Organization position paper 2009. Allergy 2009; 64 (suppl 91):1-59.
32. Calderon M A, et al., Sublingual allergen immunotherapy: mode of action and its relationship with the safety profile. Allergy 2012; 67:302-11.
33. Wang, M., et al., Reduced diversity in the early fecal microbiota of infants with atopic eczema. J Allergy Clin Immunol, 2008. 121(1): p. 129-34.
34. Bisgaard, H., et al., Reduced diversity of the intestinal microbiota during infancy is associated with increased risk of allergic disease at school age. J Allergy Clin Immunol, 2011. 128(3): p. 646-52 e1-5.
35. Abrahamsson, T. R., et al., Low diversity of the gut microbiota in infants with atopic eczema. J Allergy Clin Immunol, 2012. 129(2): p. 434-440 e2.
36. Ismail, I. H., et al., Reduced gut microbial diversity in early life is associated with later development of eczema but not atopy in high-risk infants. Pediatr Allergy Immunol, 2012. 23(7): p. 674-681.
37. Lundell, A. C., et al., Increased levels of circulating soluble CD14 but not CD83 in infants are associated with early intestinal colonization with Staphylococcus aureus. Clin Exp Allergy, 2007. 37(1): p. 62-71.
38. Kappler, J., et al., V beta-specific stimulation of human T cells by staphylococcal toxins. Science, 1989. 244(4906): p. 811-3.
39. White, J., et al., The V beta-specific superantigen staphylococcal enterotoxin B: stimulation of mature T cells and clonal deletion in neonatal mice. Cell, 1989. 56(1): p. 27-35.
40. Hu, D. L., et al., Staphylococcal enterotoxin induces emesis through increasing serotonin release in intestine and it is downregulated by cannabinoid receptor 1. Cell Microbiol, 2007. 9(9): p. 2267-77.
41. Lonnqvist A et al., Neonatal exposure to staphylococcal superantigen improves induction of oral tolerance in a mouse model of airway allergy. Eur J Immunol, 2009. 39: 447-456.
42. Matricardi, P. M., et al., Exposure to foodborne and orofecal microbes versus airborne viruses in relation to atopy and allergic asthma: epidemiological study. Bmj, 2000. 320(7232): p. 412-7.
43. Nakao, A., et al., High-dose oral tolerance prevents antigen-induced eosinophil recruitment into the mouse airways. Int Immunol, 1998. 10(4): p. 387-94.
44. Almqvist, N., et al., Serum-derived exosomes from antigen-fed mice prevent allergic sensitization in a model of allergic asthma. Immunology, 2008. 125(1): p. 21-7.
45. Proft, T. and Fraser, J. D., Bacterial superantigens. Clin Exp Immunol 2003; 133:299-306.
46. Lina G, Bohach G A, Nair S P, Hiramatsu K, Jouvin-Marche E, Mariuzza R; International Nomenclature Committee for Staphylococcal Superantigens. Standard nomenclature for the superantigens expressed by Staphylococcus. J Infect Dis. 2004 Jun. 15; 189(12):2334-6.
47. Stern A, Wold A E, Östman S; PLOS ONE, September 2013, vol. 8(9), e75594.

1. A method of enhancing the effect of allergen specific immune therapy (ASIT) in a non-human mammal suffering from hypersensitivity towards an allergen, the method comprising the steps of:
administering the allergen to the non-human mammal; and
administering a superantigen before, or with, the allergen to the non-human mammal in need thereof.

2. The method according to claim 1, wherein the non-human mammal is a dog, a cat, or a horse.

3. (canceled)

4. The method according to claim 1, wherein the superantigen and the allergen are co-administered.

5. The method according to claim 1, wherein at least one of the superantigen and the allergen is orally administered.

6. The method according to claim 5, wherein at least one of the superantigen and the allergen is sublingually administered.

7. The method according to claim 1, wherein the superantigen is administered less than 18 hours before the administration of the allergen.

8. The method according to claim 1, wherein the steps of administering the superantigen and the allergen are repeated, the subsequent administration being performed is at least 4 hours after the preceding administration but less than 2 weeks after the preceding administration.

9. The method according to claim 1, wherein the allergen is formulated for oral administration.

10. The method according to claim 1, wherein the non-human mammal is at least 6 months old.

11. The method according to claim 1, wherein said superantigen is selected from the group consisting of: SEA, SEB, SEC1, SEC2, SEC3, SED, SEE, SEG, SEH, SEI, SEJ, SEK, SEL, SEM, SEN, SEO, SEP, SER, SEQ, SEU, SEV, TSST-1, and a mixture thereof.

12. The method according to claim 11, wherein said superantigen is selected from the group consisting of: SEK, SEL, SEM, SEN, SEO, SEP, SEQ, SEU, and a mixture thereof.

13. The method according to claim 1, wherein the allergen specific immune therapy (ASIT) targets allergies selected from the group consisting of canine atopic dermatitis (CAD) and food allergy.

14. The method according to claim 1, wherein the allergen is selected from the group consisting of environmental allergens and food allergens.

15. A composition comprising a superantigen, an allergen and at least one pharmaceutical acceptable carrier or excipient.

16. The composition according to claim 15, wherein the composition is formulated for oral administration.

17. The composition according to claim 15, wherein the superantigen is selected from the group consisting of: SEA, SEB, SEC1, SEC2, SEC3, SED, SEE, SEG, SEH, SEI, SEJ, SEK, SEL, SEM, SEN, SEO, SEP, SER, SEQ, SEU, SEV, TSST-1, and a mixture thereof.

18. The composition according to claim 17, wherein the superantigen is selected from the group consisting of: SEK, SEL, SEM, SEN, SEO, SEP, SEQ, SEU, and a mixture thereof.

19. The composition according to claim 15, wherein the allergen is selected from the group consisting of environmental allergens and food allergens.

20. (canceled)

21. (canceled)

22. (canceled)

23. The method according to claim 14, wherein the allergen is selected from the group consisting of tree-pollen, grass-pollen, wood-pollen, house dust mites, mold spores, fleas, beef protein, chicken protein, pork protein, corn protein, wheat protein, soybean protein, and egg protein.

24. The method according to claim 19, wherein the allergen is selected from the group consisting of tree-pollen, grass-pollen, wood-pollen, house dust mites, mold spores, fleas, beef protein, chicken protein, pork protein, corn protein, wheat protein, soybean protein, and egg protein.

25. The method according to claim 4, wherein the superantigen and the allergen are formulated into a single composition with at least one pharmaceutically acceptable carrier or excipient.
